(19) United States
(12) Patent Application Publication
(10) Pub. No.: US 2013/0280695 A1
(43) Pub. Date: Oct. 24, 2013

(54) METHOD, DEVICE AND TEST KIT FOR MOLECULAR-BIOLOGICAL REACTIONS

(71) Applicants: Timo Hillebrand, Hoppegarten (DE); Elmara Graser, Berlin (DE); Katjana Daskalow, Berlin (DE)

(72) Inventors: Timo Hillebrand, Hoppegarten (DE); Elmara Graser, Berlin (DE); Katjana Daskalow, Berlin (DE)

(73) Assignee: aj Innuscreen GmbH, Berlin (DE)

(21) Appl. No.: 13/811,841

(22) PCT Filed: Jul. 22, 2011
(86) PCT No.: PCT/EP2011/062692

§ 371 Date: Jul. 12, 2013

(30) Foreign Application Priority Data
Jul. 23, 2010 (DE) 10 2010 038 330.9

(51) Int. Cl. C12Q 1/68

(52) U.S. Cl. CPC C12Q 1/6848
USPC 435/5; 435/289.1; 435/6.12

(57) ABSTRACT

The invention relates to a device, method and test kit for carrying out molecular-biological reactions, wherein the different components for the molecular-biological reactions are located on a solid carrier in different, spatially separated compartments prior to the start of the reaction. The carrier is preferably a porous filter disk made of polyethylene. Fields of application are the amplification of nucleic acids, for example PCR or RealTime PCR, the reverse transcription of RNA in DNA enzyme-substrate interactions or antigen-antibody interactions, or protein synthesis.

The subject matter of the invention is reagent components for performing molecular-genetic investigations, especially under field conditions. These reagent formulations are also stable during storage at room temperature. Possible areas of application are the polymerase chain reaction (PCR), reverse transcription (RT), enzyme-substrate and antigen-antibody interactions, and protein synthesis.

### BACKGROUND ART

The investigation of diagnostically relevant biological samples such as serum, plasma, blood, urine, swab samples or organ grit for detection of infectious pathogens has increased enormously in importance in recent years. Virus infections such as HIV, HCV or HBV are spreading worldwide. Furthermore, bacterial infections are also increasingly prevalent throughout the world, among other reasons as the result of incipient climatic changes. The emergence of new infectious diseases, some with fatal outcome, and an extremely high infection potential (severe acute respiratory syndrome (SARS), bird flu) is proving ever more clearly that a rapid diagnosis capable of being performed on the spot will be decisive in the future for preventing epidemics. Furthermore, diagnostic systems that are easy to handle, reliable and at the same time relatively inexpensive, especially in developing countries, will also play a significant role in fighting the spread of infectious diseases. The tests used at present, especially for the detection of viral infectious diseases (HIV, HCV, HBV, dengue fever and many others) are based mostly on invasive sampling and on performing real-time PCRs. These tests are dependent on extremely expensive instrumental prerequisites as well as on expensive reagents. Such methods can be performed only by trained specialized personnel in special-purpose laboratories.

A further main aspect relates to the performance of diagnostic tests aimed at early detection of bioterrorist attacks (smallpox, plague, anthrax). In this context it is extremely important to be able to use mobile detection systems. Some few mobile detection systems have meanwhile become available. However, their areas of application are still very limited at present. These systems are based in principle on the implementation of traditional diagnostic methods, which are used in standardized manner under laboratory conditions. After isolation of the nucleic acids, a specific amplification reaction is carried out with specific detection of the PCR products.

In this connection, however, a problem for the use of traditional laboratory-diagnostic detection methods under field conditions is that no refrigeration capacities are available under field conditions and that the extensive steps of pipetting of reaction batches (e.g. for a PCR) are also not feasible under field conditions. Usually these reactions can be performed only by specialized personnel.

The object of the invention was therefore among others to provide all critical reagent components needed for molecular-diagnostic detection methods in a form that:
1. permits very simple handling of the preparation of reaction batches and
2. can also be used without refrigeration and under ambient temperature conditions.

For molecular-genetic detection methods, a series of very different components is necessary. Examples include amplification buffers, salts, enzymes, dNTPs, nucleic acids, etc. In order to be able to assure reproducibility and functionality of the detection reaction, all components for the detection reaction, including the biomolecules, must exist stably for a relatively long time if at all possible. The shelf life of an assembly of several solutions (kit components) is only as long as that of its most sensitive component. These critical components are often biocatalysts or proteins, which depending on the application must exist in native and/or active form. Nucleotides (dNTPs), which often represent a use-limiting factor, are also highly problematic, since they become inactive very rapidly.

In order to assure a longer shelf life of such biomolecules, they are often shipped in refrigerated packages and stored at 4° C. Frequent freezing and thawing then lead to activity loss or to destruction of the biomolecule. The cause of this is the formation, in the solvent, of ice crystals, which are able to cleave the proteins. In order to prevent the formation of ice crystals, it is common practice to replace 50% of the aqueous solvent by glycerol. However, this may be added only if the activity of the biomolecules is not influenced and the glycerol does not interfere with or inhibit the subsequent applications. As alternatives to glycerol, it is also possible to use polydextrose, mannitol or sorbitol as humectants, since they replace the water functionally. In many cases, this type of stabilization is also dependent on refrigeration capability, and so degradation of the biomolecule over a longer time period cannot be ruled out. Successful storage of enzyme (preferably a Taq HOT Start Polymerase), primer, probe, dNTPs and an internal positive control is described in US Patent Application 02005 006 98 98 A1. Therein the appropriate chemicals are applied together with HEPES on a mannitol-containing bead, which is dissolved in water and then used for the PCR. A similar principle is found in nature: By virtue of the high concentration of non-reducing sugar, some organisms (microorganisms, plants, animals) are able to withstand almost complete dehydration (anhydrobiosis) and to resume normal metabolism after rehydration (Teramoto N, Sachinvala N D, Shibata M. Trehalose and trehalose-based polymers for environmentally benign, biocompatible and bioactive materials. Molecules. 2008 Aug. 21;13(8): 1773-816). The sugar is usually trehalose, a disaccharide with two glucose molecules, which is colloquially referred to as a “natural antifreeze”. It is assumed that trehalose substitutes functionally for water molecules and thus preserves the molecular integrity of the biomolecule. A corresponding patent (U.S. Pat. No. 4,891,319) describes the protection of proteins and other biological molecules by addition of trehalose to an aqueous solution. In this way, successful stabilization with preservation of the activity of the components was achieved, for example, for longer than 6 months at 40° C. for blood coagulation factors (U.S. Pat. No. 7,501,493) as well as for other molecules (Paz-Alfaro K J, Ruiz-Granados, Y G, Uribe-Carvajal S, Sampedro J G. Trehalose-mediated thermal stabilization of glucose oxidase from Aspergillus niger. J. Biotechnol. 2009 May 20; 141(3-4):130-6. Epub 2009 Mar. 25 and Morana A, Stiuso P, Colonna G, Lamberti M, Carteni M, De Rosa M. Stabilization of S-adenosyl-L-methionine promoted by trehalose. Biochim. Biophys. Acta. 2002 Nov. 14;1573(2):105-8). Primers and nucleic acids have also been stored stably with analogous success in trehalose, as described in German Patent DE 10 2006 056 790 B3. One disadvantage of sugars for stabilization during storage is that even a slight presence of water or incomplete drying of the reagents conceals the danger of contamination with bacteria or fungi.

A further possibility of longer storage stability is offered by the general removal of water. For example, biomolecules may be freeze-dried and then rehydrated before use. Even here, however, the dried biomolecules must be stored under refrigeration. In this connection, it is also worth mentioning the lyophilized particles of the Cepheid Co. (Sunnyvale, Calif.), with a size of 2.6 nm. Therein dNTPs, polymerase, magnesium chloride and buffer are combined in one particle and may be used for further applications (PCR) after addition of water and primers. For this purpose Cepheid reached back to a freeze-drying technique developed by ABAXIS Inc., known as “Orbos technology” (http://abaxis.com/about_us/history.html). A disadvantage of the drying method is the very high cost of equipment for freeze-drying.

Furthermore, it would be practical if primer and probe could also be dried. However, this would not be possible in such a form, since the mixing of primer and probe would lead to primer-dimers and other artifacts, with the result that a satisfactory subsequent PCR could no longer be assured. Furthermore, it would lead to false-positive results in numerous amplification reactions and especially reactions that couple an amplification reaction with a probe hybridization. Dimerization therefore represents a substantial problem for the stabilization of PCR batches, since several individual test tubes would be necessary in order to be able to assure an optimum course of the reaction. However, such a solution is unmanageable for field work, conceals a danger of mixups and contamination and makes it difficult to handle the overall system. In principle, it is also disadvantageous that these mixtures have a strong hygroscopic (water-attracting) effect because of the admixture of storage stabilizers, and so the activity and storage stability of the components decrease rapidly during improper storage. In order to counteract this, the dried mixture may be coated with a paraffin layer. By addition of an aqueous solution and heating, the paraffin layer melts and the dried substances dissolve and become accessible for the PCR method. This method is disclosed in EU Patent DE 10 2004 021 822 B3. Therein the reagents needed for the PCR are dried directly on the cavity where the PCR reaction also takes place.

Further storage-stable or preformulated reaction mixtures are commercially available from the GE Healthcare Co. The “Ready-to-go” product line comprises partly stabilized components for the PCR. For example, Taq polymerase, dNTPs and amplification buffer are applied on small beads. These beads are stable for a longer time period at room temperature. The disadvantage of this method is that primer and probe must be added freshly by the customer, thus making this method unsuitable for use outside a laboratory. This disadvantage is alleviated in a method developed by Klatser et al. (Klatser P R, Sjoukje Kuijper, van Ingen C W, Kolk A H J. Stabilized, Freeze-dried PCR Mix for Detection of Mycobacteria. J Clin Microb, 1998 June 36(6):1798-1800 1998). In this case, primer, buffer, dNTPs, uracil-DNA-glycosylase and polymerase are freeze-dried together with 5% trehalose. When the batches were stored at 4 or 20° C., they were still active up to one year after drying. Storage at 37° C. shortened the storage life to 3 months and at 56° C. to one week. The authors used two different polymerases. They found that a higher content of glycerol negatively influences the storage stability, since glycerol is hygroscopic. A disadvantage in this case, however, is that trehalose inhibits a series of amplification reactions, and especially reactions that couple an amplification reaction with probe hybridization. Thus such a medium is in no way universally usable. A similar method was also disclosed in German Patent DE 10 2006 056 790 B3. In that case, all components were dried in a suitable vessel and dissolved in water shortly before the PCR. Here again the dried mixture was coated by a hydrocarbon wax for better storage stability. This method was also described by Wolff et al. for a nested PCR in one test tube (Wolff C, Hörnschemeyer D, Wolff D, Kleesiek K. Single-tube nested PCR with room-temperature-stable reagents. PCR Methods Appl. 1995 4:376-379). According to German Patent DE 60 2004 002 48/5 T2, the trehalose-mediated stabilization of the reactions may be further increased by adding Prionex, a polypeptide from porcine skin collagen, as was done in this case. Therein the stability of the reagents, instead of being 30 days at 37° C., was four times as long after addition of the polypeptide as with trehalose alone.

A further method for stabilization of biomolecules consists in binding them on a shaped support. Via a coupling reaction with an amino group, the biomolecule binds stably and durably to the support material and in this way may be used for detection reactions. This method has been patented and disclosed (European Patent Application EP 0511559). A modified method, also patented, for stable immobilization of antibodies (U.S. Pat. No. 4,948,836) and oligonucleotides (German Patent DE 10053553 C2) has also been described. The drying takes place partly after incubation in a protein-sugar solution. This method is very cumbersome and time-consuming, and in this form cannot be achieved for mixing several substances, as is necessary for a PCR.

The components necessary for a molecular-genetic method (for example, PCR or reverse transcription) are nucleotide triphosphates (dNTPs), primer, probe if necessary, buffer and an enzyme (Taq or reverse transcriptase), wherein the latter catalyzed the reaction. One of the critical components for the storage stability of the individual solutions are the dNTPs, which decompose to corresponding diphosphates and monophosphates at a physiological pH (pH 7.0 to 7.5). Under physiological conditions, for example, the content of functional dNTPs already decreased by 2-3% after 10 days at 37° C. In order to counteract this, the dNTPs may be stored in the presence of an appropriate stabilizer. In particular, the stability of adenosine triphosphate has been confirmed in the presence of EDTA (pH 8.3 to 9.2), guanidine/aminoguanidine (pH 9.0 to 10.0) or glycerol/hydrogen phosphate (pH 3.7) as stabilizers (EP 0941370 B1). Prolonged stable storage without the need for a stabilizer is disclosed in Patent EP 0941370 B1. In this case the stability of the dNTPs at a pH between 8.0 and 10.0 is described, wherein the value may be adjusted both by addition of bases and by addition of buffers (tris buffer, sodium carbonate buffer, phosphate buffer).

The publications U.S. Pat. No. 3,645,853 A, DE 2800437 A1, DE 1220083 A, WO 98/37229 A1, EP 0733714 A2, EP 0823972 A1 and EP 0925113 A1 also belong to the background art.

In U.S. Pat. No. 3,645,853, a diagnostic composition and a method for detection of nitrate reduction are described. A sponge-like material is disclosed that contains at least four impregnated zones. The compartmentalization is organized such that solutions are applied individually on paper and after complete drying are then manually joined together. By virtue of its nature, compatibility does not exist. Substance is detached from one side only. Other components are not dissolved, but a direct color change takes place after mixing of the solutions on the strip.

DE 2800437 A1 mentions a device for obtaining a non-toxic atmosphere for use in culturing anaerobic microorganisms, wherein the surface area and the permeability of a membrane are selected such that the arrival velocity of the liquid and therefore also the rate of generation of the gas atmosphere is controlled.

German Unexamined Application DE 1220083 A describes sub-chambers, which are integrated into a main chamber and can be separated from one another by insertable plates.

In International Patent Application WO 98/37229 A1, a method and apparatus for a rapid hygiene test are proposed. Therein the liquid, which may include an extraction reagent, is applied on a test surface. No compartmentalization is used.

In EP 0 733 714 A2, a nucleic acid amplification method and apparatus are described. A sample is pumped from the sample application to the reaction area. The reaction area is described as preferably a continuous channel: “Although it is preferred to provide the sample area and reaction area in the form of a continuous, undivided channel as shown, it is within the scope of the present invention to provide partitions or barriers between these areas if desired. It is also within the scope of the invention to provide partitions or barriers between the decontamination zone and amplification zone of the reaction area.” However, the authors had no intention of separating the products from one another.

The subject matter of publication EP 0733714 A2 is a diagnostic device in which the release and capture medium form a two-phase chromatographic substrate. The two media are separated from one another and consist of different materials. The capture medium has no further immobilized components, but has the function of adsorption. The release medium is a hydrophilic material for retention, release and transport of the immunological material. Application takes place by capillary effects of the test solution. In this case, mixing is not systematically prevented.

EP 0 925 113 A1 reports on a device and a method for storage and distribution of biochemical reagents. Each reagent is disposed in its own space and the spaces are separated from one another. The reagent cartridge is an individual article. Reagent cartridges are combined as cassette cartridges.

The analysis of the background art therefore shows impressively that a large number of disclosures exist that relate to stabilization of reaction components for molecular-genetic detection methods. However, they always relate to solution batches, which cannot guarantee any universal use of the actual detection reaction or cannot stabilize the entire necessary reaction mixture.

### OBJECT OF THE INVENTION

The object of the invention was to eliminate the disadvantages mentioned in the background art.

### ACHIEVEMENT OF THE OBJECT

The object was achieved according to the features of the claims. According to the invention, there has been provided with the present invention a molecular-diagnostic detection method that contains all critical reagent components needed in a form that:
1. permits very simple handling of a molecular-genetic detection method and
2. can also be used universally without refrigeration under ambient temperature conditions.

In particular, the spatial separation of the components participating in the amplification or reverse transcription was not taken into consideration in any of the solutions disclosed heretofore. In contrast, the inventive means takes precisely into consideration the spatial separation of the components needed for the reactions, and in this way enables protection of the participating components from one another. Thus this approach makes impossible an unwanted start of non-specific reactions (such as formation of primer dimers, unspecific amplification under the suboptimum conditions, etc.).

The invention is described as follows:

Polymerase, primer or primer and probe needed for a PCR-based amplification reaction (standard PCR or real-time PCR) are applied on a porous filter disk (e.g. of polyethylene). The use of such a material surprisingly reveals a number of advantages. It is possible to position the components as individual components at designated locations on the filter disk and thus to deposit them in compartmentalized form. In this way the components to be combined do not come into contact with one another. This has the advantage that an undesired reaction of the components with one another may be prevented. A further advantage is that, by the penetration of the components into the pores of the filter disk, better protection against harmful environmental influences is achieved, with favorable consequences for storage at ambient temperature without activity loss. After all needed components have been applied on the porous filter disk, the components are dried (e.g. incubation at 37° C. for 1 hour) or else are lyophilized. Thereafter the filter disk is stored dry. The release of the applied reaction components takes place by feeding to the filter disk an aqueous solution that contains all further reaction components needed for the amplification reaction/detection reaction in the respective optimum concentrations (dNTPs, amplification buffer, magnesium and further additives if necessary). This aqueous solution may be prepared beforehand as a master mix and may already also contain the nucleic acid to be investigated. Preferably a detergent (e.g. an octylphenol ethoxylate or polysorbate) is added to the aqueous solution. This detergent improves the release of the components present on the filter disk. After brief incubation, the batch is transferred into a PCR test tube and the reaction is carried out.

Besides the application of a polymerase, primers or primers and probe, the needed amount of dNTPs may additionally be deposited on the filter disk in compartmentalized manner and thus spatially separated from the other reaction components. The preparation of the storage-stable filter disk as well as the storage takes place as described. The release of the applied reaction components again takes place by feeding to the filter disk an aqueous solution that contains all further reaction components needed for the amplification reaction/detection reaction in the respective optimum concentration. This aqueous solution may be prepared in advance and may already also contain the nucleic acid to be investigated. An advantage of this embodiment is that the dNTPs have also been deposited in storage-stable form on the filter disk. Thus this component no longer has to be contained in the aqueous solution for release of the reaction components. This makes it easier to carry out a detection reaction, e.g. additionally under field conditions, since the aqueous solution now contains only amplification buffer, magnesium and if necessary further additives, preferably the already mentioned detergent. Such a solution may also be stored without problems even at ambient temperature. In combination with the storage-stable filter disk, therefore, a means for achieving the goals of the invention is ideally available.

The inventive means may also be used to carry out a reverse transcriptase reaction for investigation of a target RNA. For this purpose, the reverse transcriptase, dNTPs, the concentrated buffer and the primer needed for the reaction (e.g. random primer) are deposited in compartmentalized manner on the filter disk. The detachment of the components then again takes place with an aqueous solution containing all other needed reaction components (RNA to be transcribed; if necessary a detergent and RNA-stabilizing reagents). Besides stabilizing the reagents, this method has yet a further advantage: The amount of the RNA used may be considerably increased by adding the necessary components not as an aqueous solution but instead in a form directly dissolved in the RNA to be transcribed. This increases the sensitivity of the test method.

In the case of what is known as one-step PCR (cDNA synthesis and PCR in the same test tube), the reaction buffer, the enzyme mix, the dNTPs and the primer and probe necessary for the reaction are deposited in compartmentalized form and stabilized on the filter disk. The detachment of the components takes place, as also in the case in a two-step PCR (cDNA synthesis and PCR in different test tubes), with an aqueous solution containing the RNA to be investigated.

Furthermore, the compartmentalization and storage-stable immobilization of individual reaction components may be used for different mixtures and enzyme-catalyzed reactions. It is always ideal when the needed reaction partners must be prevented from interacting with one another before a reaction.

The application of different reaction components needed for a reaction in an arrangement spatially separated from one another may be achieved on spatially separated areas of a tube, even without porous material. As in the case of the filter disk also, the individual components are brought together by washing them out with an aqueous solution immediately before the start of the reaction. In this case a minor restriction exists in the fact that the inventive filter disk offers greater protection from exogenous factors (e.g. oxygen, moisture, light).

### Definitions

By the term “molecular-biological reactions (hereinafter “reaction”), there will be understood an interaction of biological molecules. “Biological molecules” include any substances and compounds of substantially biological origin that have properties relevant in the context of scientific, diagnostic and/or pharmaceutical applications.

They include not only native molecules, as may be isolated from natural sources, but also forms, fragments and derivatives obtained therefrom, as well as recombinant forms and artificial molecules, provided they exhibit at least one property of the native molecules.

This includes the amplification, detection or transcription of nucleic acids, enzyme-substrate and antigen-antibody interactions, and protein synthesis. Examples are the PCR (polymerase chain reaction) of DNA and the RT (reverse transcription) of RNA into DNA.

By “primer” or “probe”, the person skilled in the art understands oligonucleotides, wherein primers are necessary for amplification or reverse transcription. The probes may be labeled. Labels for probes are known to the person skilled in the art.

The term “enzyme” includes polymerase and reverse transcriptase as well as other enzymes that can be used in molecular biology, such as peroxidases, dehydrogenases, phosphatases, etc.

dNTPs are deoxyribose nucleotide triphosphates. They are the building blocks added during a PCR in order to synthesize a new strand. Examples are: dATP (adenosine triphosphate, dUTP (uracil), dCTP (cytidine), dTTP (thymidine) and dGTP (guanosine).

The term “buffer” stands for reaction buffer. Appropriate reaction buffers are known to the person skilled in the art.

Examples of further known components for performing a reaction are salts and buffer substances, e.g. magnesium compounds.

The invention will be described hereinafter on the basis of exemplary embodiments. However, these do not constitute any limitation of the invention.

### EXEMPLARY EMBODIMENTS

### Exemplary Embodiment 1

### Preparation of a Compartmentalized Storage-Stable Reaction Batch and Use in a Storage Test for Amplification of a Human-Specific Target Sequence

### A. Preparation of a Compartmentalized Storage-Stable Reaction Batch

Polyethylene filter disks were used as the porous support.

The following components were added onto different zones of the filter disk (FIG. 1):

FIG. 1 shows a filter disk with different zones and PCR materials.
1 Ligand-modified Hot Start Taq DNA Polymerase stabilized with 10% sorbitol and 3.75% FCS in the final batch
2 dNTPs
3 Primer (sense)
4 Primer (antisense)

The components were applied in an amount corresponding to a 100-μL PCR batch.

The solutions were applied on the filter disk and dried for approximately 2 hours under physiological temperature conditions (37° C.). After completion of drying, the filter disks were transferred into a 2 mL screw top tube, securely sealed with a cap and stored at room temperature for 3 months, then tested after this storage time in a comparison reaction with freshly added reaction components.

B. Amplification of a Human-Specific Target Sequence with Fresh Reagents or Using the Reaction Batch Prepared According to the Invention After Storage for 3 Months

The amplification reaction was carried out using a SpeedCycler (Analytik Jena AG).

Batches:

### 1) “Fresh”

5 μL DNA (human)
10 μL amplification buffer, including magnesium chloride (10×)
2 μL dNTPs (12.5 mM, in each case)
0.7 μL primer (sense; 50 pmol)
0.74 primer (antisense; 50 pmol)
5 units Taq polymerase
Made up with water to final volume of 100 μL
2) “Storage-stable filter disk”
5 μL DNA (human)
10 μL amplification buffer, including magnesium chloride (10×)
Made up with water to final volume of 100 μL

The DNA buffer mixture was added onto the filter plate in 2 mL tubes and vortexed for 1 minute. The 100 μL of each PCR batch was distributed in 15 μL aliquots into 6 wells of a SpeedCycler plate. After completion of the amplification reaction, the amplification products were evaluated on an agarose gel (FIG. 2).

FIG. 2 shows PCR products after amplification of genomic DNA with fresh and storage-stable reagents.

M . . . DNA label
Lanes 1-6 . . . Amplification products using the filter disk coated to achieve storage stability (Batch 1)
Lanes 7-12 . . . Amplification products using freshly added reaction components (Batch 2)

The example illustrates impressively that the use of the filter disk coated to achieve storage stability does not exhibit any kind of activity loss after storage of 3 months at room temperature when compared with freshly added reaction components.

### Exemplary Embodiment 2

Preparation of a Compartmentalized Storage-Stable Reaction Batch and Use in a Storage Test for Amplification and Hybridization of a Salmonella enterica—Specific Target Sequence

The compartmentalized reagents were prepared for performing a probe-based LFA assay. During performance of the assay, it is essential that the FITC-labeled probe and the biotin-labeled primer do not form any dimerization products before the reaction. According to the invention, the compartmentalized application of the individual reaction components is intended to prevent an irregular start to the reaction and in turn to prevent the occurrence of false-positive results.

### A. Preparation of a Compartmentalized Storage-Stable Reaction Batch

Polyethylene filter disks were used as the porous support.

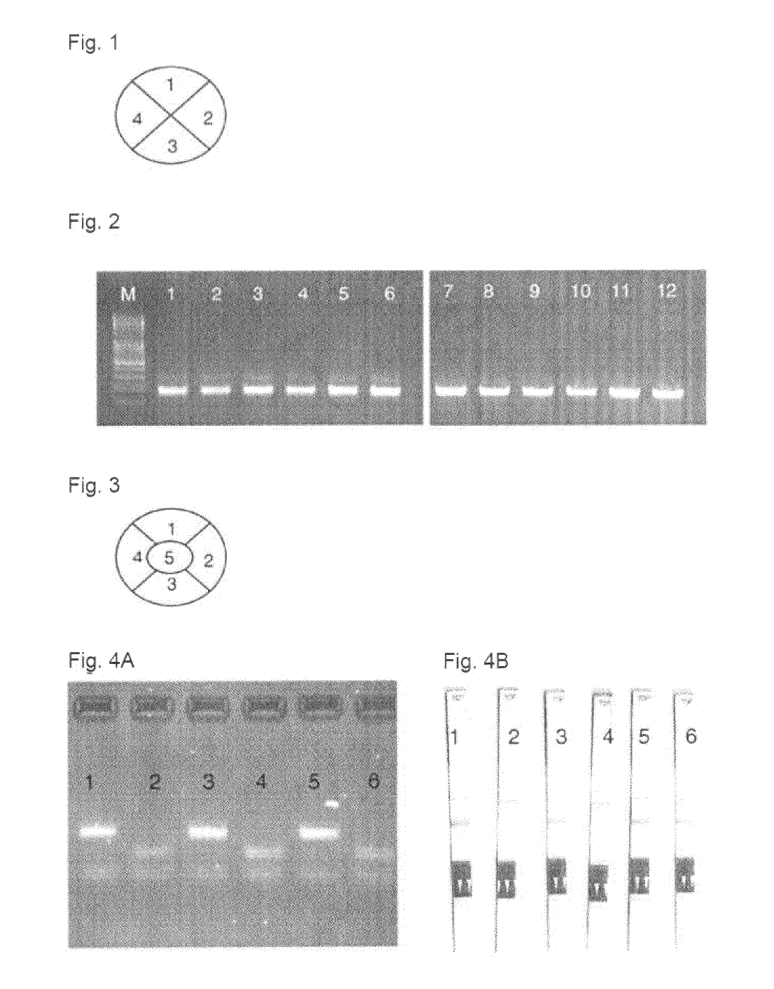

The following components were added onto different zones of the filter disk (FIG. 3):

FIG. 3 shows the overhead view of a filter disk. Compartmentalization of storage-stable components for a probe-based LFA assay.
1 dNTPs
2 FITC-labeled probe
3 Primer 1, unlabeled
4 Primer 2, biotin-labeled
5 Ligand-modified Hot Start Taq DNA Polymerase stabilized with 10% sorbitol and 3.75% FCS in the final batch

The reagents were applied in an amount corresponding to a 100-μL PCR batch. The solutions were pipetted onto the filter disk and dried for approximately 2 hours under physiological temperature conditions of 37° C.

After completion of drying, the filter disks were placed in a 2 mL screw top tube, securely sealed with a cap and stored at RT for 3 months, then tested in a comparison reaction with freshly added reaction components.

### B. Amplification of a Human-Specific Target Sequence

The amplification reaction was carried out using a SpeedCycler (Analytik Jena AG).

### Batches:

### 1) “Fresh”

10 μL amplification buffer, including magnesium chloride (10×)
2 μL dNTPs (12.5 mM, in each case)
0.35 μL primer (unlabeled; 50 pmol)
0.7 μL primer (biotin-labeled; 50 pmol)
0.7 μL primer (FITC-labeled; 50 pmol)
5 units Taq polymerase
Made up with water to final volume of 100 μL

2×15 μL aliquots of the batch were removed by pipette as the negative control, 5 μL aliquots of S. enterica DNA were added to the rest of the master mix, and from this 2×15 μL aliquots of the PCR batch were added into the well of a PCR plate for the SpeedCycler.

2) “Storage-stable filter disk”
10 μL amplification buffer, including magnesium chloride (10×)
Made up with water to final volume of 100 μL

The DNA buffer mixture was added onto the filter plate in 2 mL tubes, sealed with a cap and vortexed for 1 minute. 2×15 μL aliquots of the batch were removed by pipette as the negative control, 5 μL aliquots of S. enterica DNA were added to the rest of the master mix, and from this 2×15 μL aliquots of the PCR batch were added into the well of a PCR plate for the SpeedCycler.

3) “Fresh+primer/probe mix”

In this batch, primer and probe were mixed together in the given amounts (see “fresh” batch) 2 hours before the reaction and stored at room temperature in order to favor dimerization. 2×15 μL aliquots of the batch were removed by pipette as the negative control, 5 μL aliquots of S. enterica DNA were added to the rest of the master mix, and from this 2×15 μL aliquots of the PCR batch were added into the well of a PCR plate for the SpeedCycler.

After completion of the amplification/hybridization reaction by means of a SpeedCycler, the respective 2 equal batches were mixed together. One half was applied on an agarose gel (FIG. 4A). The other half was applied on a lateral flow strip (FIG. 4B).

FIG. 4 shows an amplification-hybridization reaction (RAH technology) after evaluation of the PCR batch on the gel (A) and on an LFA (B).

1 . . . Batch 1 (fresh), positive PCR; 2 . . . Batch 1, negative control; 3 . . . Batch 2 (storage-stable filter disk), positive PCR; 4 . . . Batch 2, negative control; 5 . . . Batch 3 (fresh+primer/probe mix), positive PCR; 6 . . . Batch 3, negative control

This example shows that a long incubation of primer and probe at room temperature may lead to formation of dimers between the labeled probe and the labeled primer. This phenomenon is indeed not visible on the agarose gel (the negative control remains negative) but leads to false-positive test results on a lateral flow strip, where all doubly labeled dimers are visible and thus convey a false-positive test result. In the case of compartmentalized immobilization, the needed reaction components can already be brought together in the specified amounts. By virtue of the spatial separation of the components, however, the possibility of an undesired reaction start or of dimerization does not exist.

### Exemplary Embodiment 3

Preparation of a Compartmentalized Storage-Stable Reaction Batch and Use Thereof for One-Step cDNA Synthesis and Amplification of an Influenza A Virus.

### A. Preparation of a Compartmentalized Storage-Stable Reaction Batch

Polyethylene filter disks were used as the porous support.

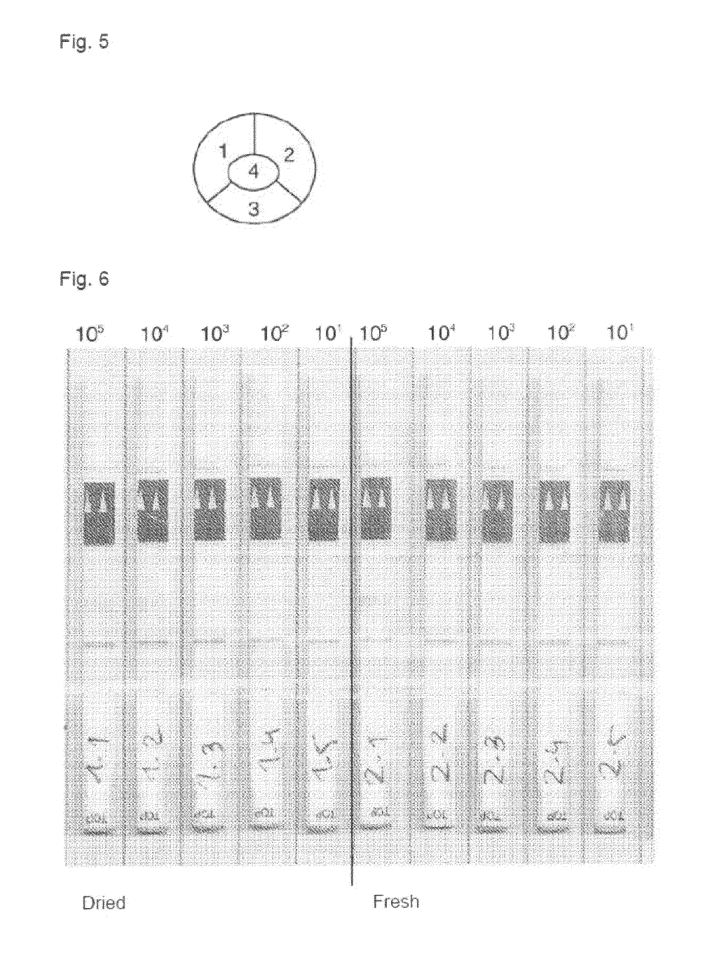

The following components were added onto different zones of the filter disk (FIG. 5):

FIG. 5 shows the overhead view of a filter disk. Compartmentalization of storage-stable components for a one-step PCR.

1. FITC-labeled probe
2. Primer 1, unlabeled
3. Primer 2, biotin-labeled
4. Affinityscript enzyme mix for one-step RT-PCR (stabilized if necessary)
5. Furthermore, buffer containing dNTPs was dried on the bottom of the test tube (Herculase® II RT-PCR 2× Mastermix, Agilent)

The chemicals were applied in an amount corresponding to a 100-μL batch. The solutions were applied on the filter disk or on the bottom of a 2-ml screw top tube, sealed with a screw cap and dried for approximately 2 hours under physiological temperature conditions of 37° C.

After completion of drying, the filter disks were placed in a 2 mL screw top tube, stored at RT for 3 months and tested in a comparison reaction with freshly added reaction components.

### B. One-Step RT-PCR of an Influenza A-Specific Target Sequence

The amplification reaction was carried out using a SpeedCycler (Analytik Jena AG). RNA samples:

A dilution series of cultured Influenza A viruses was prepared on RNA samples. The samples were diluted such that 12 μL RNA corresponded respectively to 10⁵, 10⁴, 10³, 10² and 10¹ virus particles.

### Batches:

### WO 2012/010708 PCT/EP2011/062692

### 1) “Fresh”

12 μL virus RNA

### 50 μL Herculase® II RT-PCR 2× Mastermix (Agilent)

0.35 pit primer (unlabeled; 50 pmol)
0.7 μL primer (biotin-labeled; 50 pmol)
0.74 primer (FITC-labeled; 50 pmol)
1 μL Affinityscript enzyme mix (Agilent)
Made up with water to a final volume of 100 μL
2) “Storage-stable filter disk”
12 μL virus RNA
Made up with water to final volume of 100 μL

The RNA solution was added onto the filter plate in 2 mL tubes and vortexed for 1 minute.

After completion of the one-step RT-PCR and subsequent hybridization of the probe by means of a SpeedCycler, the reaction products were applied on lateral flow strips (FIG. 6).

FIG. 6 shows different virus dilutions were amplified with freshly prepared components (fresh) or storage-stable components (dried) on a filter disk in a one-step RT-PCR and detected on a lateral flow strip.

The example illustrates that the use of the filter disk coated to achieve storage stability does not exhibit any kind of activity loss compared with freshly added reaction components, even in a one-step RT-PCR reaction.

1. A device for performing a molecular-biological reaction, comprising:
individual reaction components; and
a solid support material;
wherein individual reaction components needed for a reaction are present at the same reaction site, but before the reaction are spatially separated from one another on said solid support material, so that an unwanted interaction of the individual reaction components before the reaction start is impossible.

2. The device according to claim 1, wherein the molecular-biological reactions are:
a) amplification of nucleic acids, e.g. PCR or real-time PCR, or
b) reverse transcription of RNA into DNA, or
c) enzyme-substrate interactions, or
d) antigen-antibody reactions, or
e) protein syntheses.

3. The device according to claim 1, wherein the solid support material is a porous support material.

4. The device according to claim 1, wherein the solid support material has a surface on which the individual reaction components are applied in spatially separated manner.

5. The device according to claim 1, wherein the support comprises of polyethylene.

6. The device according to claim 5, wherein
f) primer and enzyme, or
g) primer, enzyme and probe or
h) primer, enzyme, probe and dNTPs, or
i) primer, enzyme, probe, dNTPs and buffer or
j) enzyme and coenzyme or
k) two or more different antibodies
are present on the support in different spatially separated compartments.

7. The device according to claim 6, wherein at least one of the said components f) to k) for the reaction is immobilized in different compartments on the support.

8. The device according to claim 7, wherein the components f) to k) are stabilized on the support by removal of water or substitution of water.

9. A method for performing a reaction, comprising:
a) preparing a solid support;
b) adding a liquid containing a biomolecule to be detected and optionally other substances needed for said reaction; and
c) incubating and performing said reaction.

10. The method according to claim 9, wherein the liquid is pure water, a solution, a detergent-containing solution, a solution containing the sample to be tested, or a solution containing a further reaction component.

11. The method according to claim 10, wherein nonionic surfactants are used as the detergent.

12. The method according to claim 9, wherein, for the case that the support does not contain all substances needed for the reaction, these components are present in the solution.

13. A molecular-diagnostic test kit, comprising:
a solid support comprising individual reaction components; and
a liquid containing a biomolecule.

14. The test kit according to claim 13, further comprising a component for performing and evaluating a molecular-diagnostic reaction.

15. A method of performing a molecular-diagnostic test, comprising:
contacting the liquid of the test kit according to claim 13 with the solid support.

16. The test kit according to claim 13, wherein the liquid is pure water, a solution, a detergent-containing solution, a solution containing the sample to be tested, or a solution containing a further reaction component.

17. The test kit according to claim 16, wherein nonionic surfactants are used as the detergent.

18. The test kit according to claim 13, wherein the solid support is a porous support.

19. The test kit according to claim 13, wherein the solid support has a surface on which the reaction components are applied in spatially separated manner.

20. The test kit according to claim 13, wherein the support comprises polyethylene.
